(19) United States
(12) Patent Application Publication
(10) Pub. No.: US 2026/0315733 A1
(43) Pub. Date: Oct. 15, 2026

(54) SHEATH WITH FLEXIBLE CIRCUIT

(71) Applicant: Boston Scientific Scimed, Inc., Maple Grove, MN (US)

(72) Inventors: Esme Bonnell, Toronto (CA); Miles Montgomery, East Gwillimbury (CA); Brian Ma, Etobicoke (CA); Miriam Lutes, Ottawa (CA); Luke Keaveney, Georgetown (CA); Jastyn Balasubramaniam, Brampton (CA)

(21) Appl. No.: 19/643,422

(22) Filed: Apr. 9, 2026

Related U.S. Application Data
(60) Provisional application No. 63/785,924, filed on Apr. 9, 2025.

(51) Int. Cl. A61M 25/00; A61B 5/287; A61B 18/14; A61M 25/01; A61M 25/06

(52) U.S. Cl. CPC A61M 25/0012; A61M 25/005; A61B 5/287; A61B 18/1492; A61M 2025/0166; A61M 2025/0681

(57) ABSTRACT

A sheath comprises an elongated shaft defining a major lumen along a longitudinal axis and has a distal section opposite a proximal section. The shaft includes a flexible circuit. The flexible circuit has a longitudinally extending electrically insulative substrate configured in a serpentine route, an electrically conductive lead trace disposed on the electrically insulative substrate along the serpentine route, and an electrode exposed on a radially outward surface of the shaft in the distal section and electrically coupled to the lead trace. The flexible circuit extends from the proximal section to the distal section of the shaft.

### CROSS REFERENCE TO RELATED APPLICATION

The present application claims priority to and the benefit of U.S. Provisional Patent Application No. 63/785,924 filed Apr. 9, 2025, the disclosure of which is incorporated herein in its entirety.

### TECHNICAL FIELD

The present disclosure relates to medical devices for catheterization procedures, such as medical devices for electrophysiological procedures. More specifically, the present disclosure relates to catheter elements, such as sheaths, and methods for manufacturing sheaths.

### BACKGROUND

Various medical fields use different types of catheters to achieve access to a physiological site in medical procedures. For instance, electrophysiological procedures involve guiding catheter elements into the heart and tracking the location of the catheter elements with respect to the heart. Catheter ablation is minimally invasive electrophysiological procedure to treat a variety of heart conditions such as supraventricular and ventricular arrhythmia. Example catheter elements used in catheter ablation can include mapping catheters, ablation catheters, guiding sheaths, dilators, and other medical tools, which can be referred to as catheter elements in this disclosure. Electrophysiological procedures can involve the visualization of the heart, heart activity, and the position of the catheter elements within the heart. A common visualization system involves the use of fluoroscopy, which can expose the patient and clinician to ionizing radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheter elements for positioning and manipulation. Catheter elements used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheter elements include magnetic sensors. Impedance-based catheter elements, such as sheaths, use electrodes in the presence of electric fields to track the sheaths.

### SUMMARY

In Example 1, a sheath, comprising: an elongated shaft defining a major lumen along a longitudinal axis and having a distal section opposite a proximal section, the shaft including a flexible circuit; the flexible circuit having a longitudinally extending electrically insulative substrate configured in a serpentine route, an electrically conductive lead trace disposed on the electrically insulative substrate along the serpentine route, and an electrode exposed on a radially outward surface of the shaft in the distal section and electrically coupled to the lead trace, the flexible circuit extending from the proximal section to the distal section of the shaft.

In Example 2, the sheath of Example 1, wherein the lead trace is copper tracing laminated on the substrate.

In Example 3, the sheath of any of Examples 1 and 2, wherein the substrate is a flexible and stretchable thermoplastic polyurethane.

In Example 4, the sheath of any of Examples 1-3, wherein the lead trace is disposed on an inner substrate layer along a portion of the flexible circuit.

In Example 5, the sheath of Example 4, wherein the lead trace is disposed between a plurality of substrate layers including the inner substrate layer and an outer substrate layer along a portion of the flexible circuit.

In Example 6, the sheath of any of Examples 1-5, wherein the flexible circuit includes an interface pad in the proximal section.

In Example 7, the sheath of Example 1, wherein the shaft includes an elongated and electrically insulative cover member having the radially outward surface.

In Example 8, the sheath of Example 7, wherein the shaft includes an elongated braided support member radially disposed underneath the cover member.

In Example 9, the sheath of Example 8, wherein the braided support member includes conductive fibers.

In Example 10, the sheath of any of Examples 8 and 9, wherein the shaft includes an elongated and electrically insulative liner layer disposed radially underneath the braided support member, the liner layer having an inner wall defining the major lumen.

In Example 11, the sheath of any of Examples 7-10, wherein the cover member includes an overlayer defining the radially outward surface and inner layer radially underneath the overlayer, the flexible circuit disposed radially between the overlayer and the underlayer

In Example 12, the sheath of Example 11, wherein the cover member is skived to expose the electrode on the radially outward surface.

In Example 13, the sheath of any of Examples 7-10, wherein the flexible circuit is adhered to the cover member.

In Example 14, the sheath of any of Examples 1-13, wherein the serpentine route is a curvilinear serpentine route.

In Example 15, the sheath of Example 1, wherein the sheath includes a dilator.

In Example 16, a sheath, comprising: an elongated shaft defining a major lumen along a longitudinal axis and having a distal section opposite a proximal section, the shaft including a flexible circuit; the flexible circuit having a longitudinally extending electrically insulative substrate configured in a serpentine route, an electrically conductive lead trace disposed on the electrically insulative substrate along the serpentine route, and an electrode exposed on a radially outward surface of the shaft in the distal section and electrically coupled to the lead trace, the flexible circuit extending from the proximal section to the distal section of the shaft.

In Example 17, the sheath of Example 16, wherein the lead trace is copper tracing laminated on the substrate.

In Example 18, the sheath of Example 16, wherein the substrate is a flexible and stretchable thermoplastic polyurethane.

In Example 19, the sheath of Example 16, wherein the lead trace is disposed on an inner substrate layer along a portion of the flexible circuit.

In Example 20, the sheath of Example 19, wherein the lead trace is disposed between a plurality of substrate layers including the inner substrate layer and an outer substrate layer along a portion of the flexible circuit.

In Example 21, the sheath of Example 16, wherein the flexible circuit includes an interface pad in the proximal section.

In Example 22, the sheath of Example 16, wherein the shaft includes an elongated and electrically insulative cover member having the radially outward surface.

In Example 23, the sheath of Example 22, wherein the shaft includes an elongated braided support member radially disposed underneath the cover member.

In Example 24, the sheath of Example 23, wherein the braided support member includes conductive fibers.

In Example 25, the sheath of Example 23, wherein the shaft includes an elongated and electrically insulative liner layer disposed radially underneath the braided support member, the liner layer having an inner wall defining the major lumen.

In Example 26, the sheath of Example 22, wherein the cover member includes an overlayer defining the radially outward surface and inner layer radially underneath the overlayer, the flexible circuit disposed radially between the overlayer and the underlayer.

In Example 27, the sheath of Example 26, wherein the cover member is skived to expose the electrode on the radially outward surface.

In Example 28, the sheath of Example 22, wherein the flexible circuit is adhered to the cover member.

In Example 29, the sheath of Example 16, wherein the serpentine route is a curvilinear serpentine route.

In Example 30, the sheath of Example 16, wherein the sheath includes a dilator.

In Example 31, a sheath, comprising: an elongated shaft defining a major lumen along a longitudinal axis and having a distal section opposite a proximal section, the shaft including a flexible circuit and having a deflectable region and a non-deflectable region; the flexible circuit having a longitudinally extending electrically insulative substrate configured in a straight route along the non-deflectable region and a serpentine route along the deflectable region, an electrically conductive lead trace disposed on the electrically insulative substrate along the serpentine route, and an electrode exposed on a radially outward surface of the shaft in the distal section and electrically coupled to the lead trace, the flexible circuit extending from the proximal section to the distal section of the shaft.

In Example 32, the sheath of Example 31, wherein the lead trace is copper tracing laminated on the substrate, the copper tracing having a variable thickness along the deflectable and non-deflectable routes.

In Example 33, the sheath of Example 31, wherein the shaft includes an elongated braided support member radially disposed underneath the cover member, the braided support member including conductive fibers, and wherein the shaft includes an elongated and electrically insulative liner layer disposed radially underneath the braided support member, the liner layer having an inner wall defining the major lumen.

In Example 34, a method for forming a sheath, the method comprising: forming an elongated shaft defining a major lumen along a longitudinal axis and having a distal section opposite a proximal section; forming a flexible circuit having a longitudinally extending electrically insulative substrate configured in a serpentine route, an electrically conductive lead trace disposed on the electrically insulative substrate along the serpentine route; and an electrode exposed on a radially outward surface of the shaft in the distal section and electrically coupled to the lead trace, and attaching the flexible circuit to the shaft, the flexible circuit extending from the proximal section to the distal section of the shaft.

In Example 35, the method of Example 34, wherein the shaft includes an elongated and electrically insulative cover member having the radially outward surface, and including skiving the cover member to expose the electrode on the radially outward surface.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2A is a side view schematic diagram illustrating an example sheath constructed according to the present disclosure that can be used with the example electrophysiology system. of FIG. 1.

FIG. 2B is a cross-sectional end view schematic diagram illustrating an embodiment of the sheath of FIG. 2A.

FIG. 2C is a cross-sectional end view schematic diagram illustrating another embodiment of the sheath of FIG. 2A.

FIG. 3A is a plan view schematic diagram illustrating an embodiment of a flexible circuit of the sheath of FIG. 2A.

FIG. 3B is a side view schematic diagram illustrating the flexible circuit of FIG. 3A.

FIG. 4 is a schematic diagram illustrating an element of the flexible circuit of FIG. 3A.

FIG. 5 is a schematic diagram illustrating a deflected sheath.

FIG. 5A is a schematic diagram illustrating an embodiment of a deflected sheath.

FIG. 5B is a schematic diagram illustrating another embodiment of a deflected sheath.

FIG. 5C is a schematic diagram illustrating still another embodiment of a deflected sheath.

FIG. 6A is a cross-sectional longitudinal view of a schematic diagram illustrating an embodiment of a sheath in a manufacturing stage.

FIG. 6B is a cross-sectional longitudinal view of a schematic diagram illustrating the embodiment of the sheath of FIG. 6A in another manufacturing stage.

FIG. 7A is a cross-sectional longitudinal view of a schematic diagram illustrating an embodiment of a sheath in a manufacturing stage.

FIG. 7B is a cross-sectional longitudinal view of a schematic diagram illustrating the embodiment of the sheath of FIG. 7A in another manufacturing stage.

FIG. 8 is a cross-sectional longitudinal view of a schematic diagram illustrating still an embodiment of a sheath.

FIG. 9A is a plan view schematic diagram illustrating another embodiment of a flexible circuit of the sheath of FIG. 2A.

FIG. 9B is a plan view schematic diagram illustrating another embodiment of a flexible circuit of the sheath of FIG. 2A.

FIG. 9C is a plan view schematic diagram illustrating another embodiment of a flexible circuit of the sheath of FIG. 2A.

FIG. 9D is a plan view schematic diagram illustrating another embodiment of a flexible circuit of the sheath of FIG. 2A.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

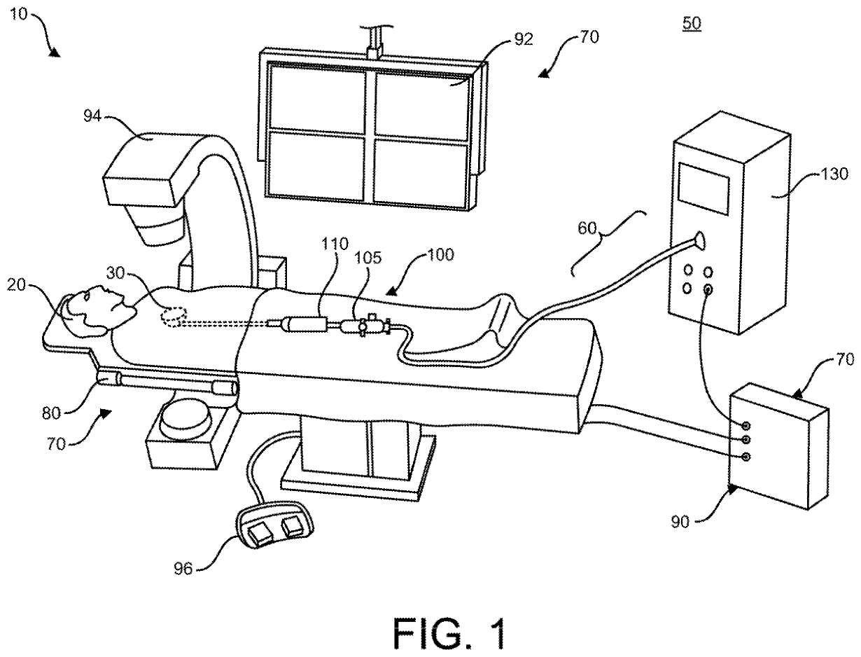

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which in the example includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheter elements. For instance, a catheter defines a longitudinal axis that passes through a centroid of a cross section of the catheter element, such as the centroid of a cross section of a shaft of catheter 105 or a centroid of a cross section of a main lumen of a sheath 110. In the example, the catheter 105 is disposed within the sheath 110. The catheter 105 and sheath 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative embodiment, the EAM system 70 can include the OPAL™ HDx mapping system (formerly available under the trade designation RHYTHMIA™ HDx mapping system) marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated embodiment, the device tracking is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter and associated sheath 110, or both. In these examples, the location sensing elements can constitute tracking electrodes on the tracked sheath that generate outputs received and processed by the mapping and navigation controller 90 to track the location of the various location sensing electrodes within the localization volume. For instance, an impedance tracking methodology can employ the use of a patch electrode (not shown) attached to the patient's body, a current or impedance based can be determined between the tracking electrode on the catheter and the patch electrode and the sheath and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter, sheath or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters and sheaths include tracking electrodes disposed on the deflectable portions of the respective shafts. Multiple tracking electrodes can be employed on the deflectable portions of the catheters and sheaths for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the sheath such as a conductive braided member along the length of the shaft. Additionally, precise placement of the tracking electrode on the shaft is desired. For instance, the tracking electrode can be radiopaque or echogenic, and clinicians can use the tracking electrode to visualize placement of the sheath while using fluoroscopy or ultrasound. Also, electrode location with respect to the catheter elements and interelectrode spacing are programmed parameters in several tracking and mapping software programs, and three-dimensional reconstruction and modeling is performed with electrode spacing as a constraint in the modeling curve. Thus, the design and implementation of sheath using tracking electrodes include lumens with ready access to carry conductor leads.

Sheaths in electrophysiological procedures primary introduce and guide other catheters to within the anatomy. Unlike many diagnostic or ablation catheters used in electrophysiological procedures, sheaths include a large portion of the diameter taken up with a major lumen suitable for receiving diagnostic catheters, ablation catheters and other tools. Typical sheaths incorporate minor lumens along the shaft to carry wires that connect to the electrodes. The lumens are often disposed below or above braided regions of the shaft, which increase components and add to the bulk of the wall of the shaft or increase manufacturing complexity. The minor lumens can affect the bending and other mechanical properties of the shaft as well as the outer diameter of the shaft, which are limited via anatomical constraints.

With increasing number of electrodes as well as other electrical devices disposed at the distal portion of the shaft, a challenge exists to maintain desirable mechanical properties as well as a limited outer diameter of the shaft. As a deflectable region of the shaft bends, the exterior of the deflectable region is stretched, and the interior of the region is compressed. With the use of stiff wire and lumen materials, the elements of the shaft buckle or break when stretched or compressed, which results in electrical failures or protrusions that manifest as wrinkles or sharp edges on the exterior of the sheath. Attempts to address these issues have removed the minor lumens from the shaft and the wires or leads are firmly affixed to the braided member, cover member, or a combination of both. Issues with these approaches include that the wires or leads provide little to no degrees of freedom for bending of the shaft, which creates problems in actuating steering mechanisms. Further, the wires and leads are under high stress during bending of the shaft, which increases the likelihood that the leads will break during a procedure, and the visualization of the distal tip will be lost. Further, the typical manufacturing process is time consuming and complex that involves ring on wire welds, wire feeding, and coiling of lumens. In addition, shafts are prone to electrical shorts between the electrodes and the braid wire, welds breaking, and wrinkling of the electrode lumens during deflections.

This disclosure is directed to catheter elements, such as sheaths, and methods for assembling sheaths that include an elongated shaft defining a major lumen along a longitudinal axis and having a distal section opposite a proximal section. The shaft includes layers such as a cover member, liner layer, or a support member. a flexible circuit, such as a flexible printed circuit, is disposed along the side of the shaft. The flexible circuit includes an electrically insulative substrate, an electrically conductive lead trace disposed on the electrically insulative substrate, and an electrode exposed on a radially outward surface of the signal layer in the distal section. The electrically insulative substrate and lead traces extend in a serpentine route from the proximal section to the distal section. The serpentine route of the insulative substrate and the lead traces is implemented as a strain relief for steerable wire to address continuity, isolation, and sheath surface roughness failures. As an additional benefit, the flexible circuit significantly reduces manufacturing time. In one embodiment, the serpentine route is disposed across a deflectable region of the sheath to avoid deflection induced wire breaks, buckling and wrinkling. During manufacture, the flexible circuit is attached to the sheath prior a cover member such as a polymer extrusion and the electrodes are ablated after the cover member is attached, or the flexible circuit is disposed on the outside of the cover member. In one embodiment, the substrate is a flexible, stretchable material, and the curvilinear serpentine route of the lead trace allows for bending and steering a reduced likelihood of breaking and affecting signal integrity. The signal layer is dedicated to transmitting the electrical signals and does not depend on overall composite design of the shaft, which allows for independent modification of signal pathways and number of electrodes without affecting the mechanical properties of the sheath. This process replaces the addition of electrode lumens, coiling, hole drilling, and wire feeding, which results in a simplification of the manufacturing process and time savings.

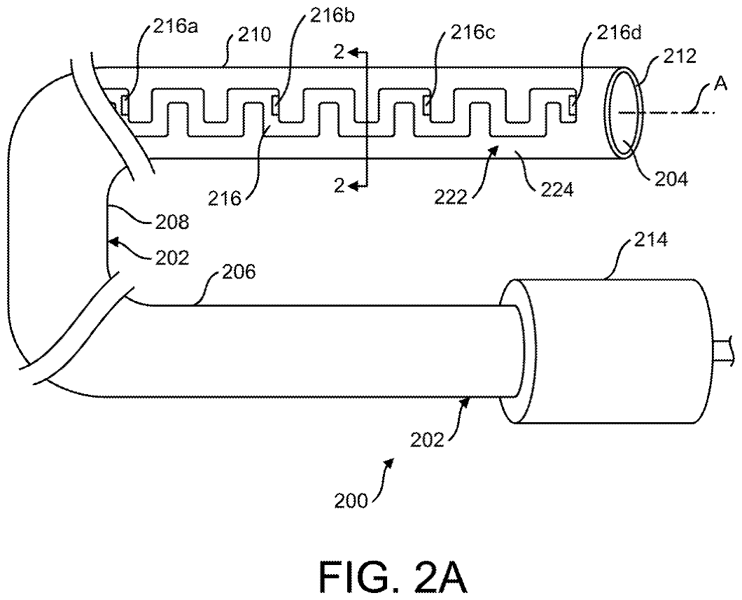

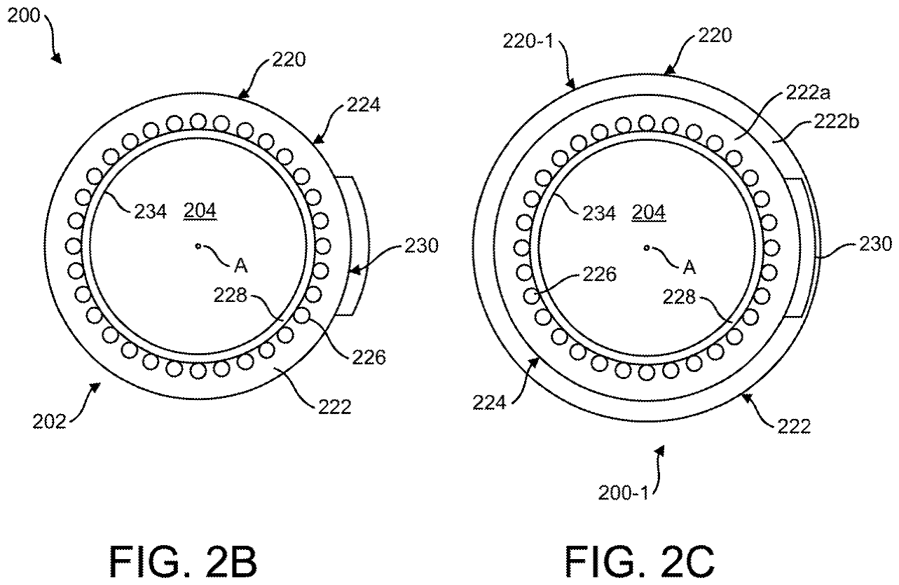

FIGS. 2A and 2B illustrates an embodiment of a catheter element 200, such as a sheath, that can be used in the example clinical setting 10 in catheter assembly 100. For instance, sheath 200 can be further configured as a guide sheath, dilator, or other flexible, deflectable medical tool that can be tracked via an impedance tracking system such as EAM system 70. In one configuration, the sheath 200 corresponds with catheter sheath 110 of FIG. 1. FIG. 2A illustrates a schematic side view of the sheath 200. The sheath 200 includes an elongated shaft 202, such as an elongated and flexible shaft 202 defining a longitudinal axis A. The shaft 202 also defines a major lumen 204 along the longitudinal axis A and has a proximal section 206, an intermediate section 208, and a distal section 210 opposite the shaft 202 from the proximal section 206. The distal section 210 includes a distal tip section 212. The proximal section 206 can be coupled to a handle 214 proximal to the shaft 202. In some embodiments, the proximal section 206 or handle 214 can include a hemostatic valve that can be coupled to a source of irrigation fluid and a port to receive catheter elements within the major lumen 204. In some embodiments, the distal tip section 212 can be configured as a dilator tip.

Referring to FIGS. 2A and 2B together, the sheath 200 includes components disposed along the shaft 202. For instance, the sheath 200 includes components disposed on the distal section 210. In the illustrated embodiment, the sheath 200 includes a tracking electrode 216, such as a plurality of exposed tracking electrodes 216a . . . 216n, on the distal section 210 of the shaft 202. The illustrated embodiment of FIG. 2A includes four tracking electrodes 216a, 216b, 216c, 216d. Each tracking electrode 216a . . . 216n is electrically coupled to a respective lead conductor extending along the shaft 202 and terminated at the proximal section 206. In the example, the tracking electrodes 216a . . . 216n are configured for use with an impedance-based tracking system to detect the position of the shaft 202, such as the distal tip section 212. In some embodiments, the tracking electrodes 216a . . . 216n are also radiopaque. In some embodiments, the distal section 210 can include other components disposed on or within the shaft 202, such as pull rings having steering wires disposed along minor lumens in the shaft extending to the proximal section 206 and various other sensors, such as a magnetic sensor, temperature sensor, a gyroscopic sensor, and an accelerometer within the distal section 210 of the shaft and mechanically and electrically coupled to leads disposed along the shaft and terminated at the proximal section 206. In some embodiments, the shaft 202 includes an irrigation conduit extending from the distal section 210 to the proximal section to supply an irrigation fluid to, for instance, the distal tip section 212. The proximal section 206 or the handle 214 in embodiments includes an electrical connection that can be coupled to an impedance-based tracking system, such as EAM system 70. The proximal section 206 or the handle 214 in embodiments includes various controls to operate the steering wires and other connections to other sensors and provide fluid flow.

FIG. 2B illustrates a cross section of an embodiment of the shaft 202 in the distal section 210 taken at lines 2-2. FIG. 2B illustrates an embodiment of the layers 220 and does not show pull wire lumens or the electrodes 216 for clarity. Referring to FIGS. 2A and 2B together, the sheath 200 includes a plurality of concentric layers 220 disposed over all of or some of the shaft 202 and along longitudinal axis A, such as a cover member 222 forming an outer surface 224 of the sheath 200, and additional layers underneath the cover member 222 such as a support member 226 and liner layer 228, that extend along the shaft 202 and are coaxial with the major lumen 204. The flexible circuit 230 is disposed on the outer surface 224 of the cover member 222 and extends longitudinally along the shaft 202. In this embodiment, the flexible circuit 230 is attached to the cover member 222 after the cover member 222 is formed on the shaft 200. In some embodiments, the flexible circuit 230 is adhered to the cover member 222, such as adhered with a pressure sensitive adhesive. In other embodiments, the flexible circuit 230 is heat staked to the cover member 22. Other configurations are contemplated. In some embodiments, the shaft 202 does not include one or more of the cover member 222, support member 226, and liner layer 228. In various embodiments, the support member 226 is a flexible braid (e.g., stainless steel braid or a high-strength polymer braid, or braid formed from a combination of materials), a coiled shaft, a hypotube (e.g., a laser cut hypotube), or a high-density polyethylene (HDPE) tube. In some embodiments, the shaft 202 can include a liner layer 228 radially underneath the support member 226 and coaxial with the main lumen 204. In other embodiments, the liner layer 228 is the support member 226, and the shaft 202 does not include a braided member or other. The liner layer 228 can define an inner wall 234 of the main lumen 204. In examples, the liner layer 228 can be a thin wall constructed of polytetrafluoroethylene (PTFE). In some embodiments, the flexible circuit 230 or support member 226 includes features that form the inner wall 234 rather than the liner layer 228. In some embodiments, the cover member 222 can be formed as a coating of an electrically insulative reflowable plastic or thermoplastic material. In one embodiment, the liner layer 228 is formed over a manufacturing mandrel to create the major lumen 204. In one configuration, the flexible circuit 230 is proud on the cover member 222. In another configuration, the flexible circuit 230 is flush with the cover member 222, or not proud of the cover member 222. In another configuration, the flexible circuit 230 is submerged within the cover member 222. In still another configuration, the flexible circuit 230 is disposed on the support member 226.

The support member 226 is illustrated in the embodiment as a braided member 226, which can provide characteristics to the sheath 200 such as reduced kinking, wrinkling, or buckling of the shaft 202 and can provide enhanced balance for pushability, deflectability, and torque transmission such as during rotation about the longitudinal axis A. In the illustration, the braided member as the support member 226 is constructed from a woven fabric or layer of braided strands or fibers that form interstitial spaces between the fibers. The braided member as the support member 226 can further be characterized by the warp and weft and bias of the fibers as well as picks per inch. For instance, in some embodiments, the picks per inch can remain generally uniform along the entire longitudinal length of the braided member as the support member 226. In some embodiments, the picks per inch can vary along portions of the longitudinal length of the braided member 226. The braided member as the support member 226 can be constructed from fibers that include stainless steel fibers, such as electrically conductive fibers, or high strength polymer fibers, or as layers of different materials. In some examples, the support member 226 can include more than one braided member, such as an innermost braided member and an outermost braided member. The braided member 226 is braided over the liner layer 228 or, in the case of braid transfer, the braided member 226 is slid over the liner layer 228.

The cover member 222 is disposed on the support member 226 and provides an outer surface 224. In some embodiments, the cover member 222 can be formed as a coating of an electrically insulative reflowable plastic or thermoplastic material that extends over the support member 226 and seals underlying components of the shaft 202. For instance, the coating can seep by reflowing over braided material of the support member 226 such as over the fibers and into the interstitial spaces of the braided material of the support member 226. The cover member 222 can also be applied in via extrusion. In one example, the cover member 222 is a polyether block amide and, in some examples, is available under the trade designations PEBAX from Arkema, S.A., and VESTAMID E from Evonik Industries, AG. Electrodes 216 are exposed on the cover member 222.

FIG. 2C illustrates a cross section of another embodiment of the shaft 202-1 in the distal section 210 in which like parts are given like reference numbers. In this example, the sheath 200-1 includes a plurality of concentric layers 220 disposed over all of or some of the shaft 202C and along the longitudinal axis, such as a cover member 222 forming an outer surface 224 of the sheath 200-1, and additional layers underneath the cover member 222 such as a support member 226 and liner layer 228, that extend along the shaft 202 and are coaxial with the major lumen 204. In the illustrated example, the cover member 222 includes a plurality of cover member layers, such as inner cover layer 222a and outer cover layer 222b. In the illustrated example, the inner cover layer 222a is disposed on an outer surface of the support member 226, although the inner cover layer 222a could be disposed on sub cover layers that are disposed on the support member or radially underneath the inner cover layer 222a. The outer cover layer 222b is disposed on an outer surface of the inner cover layer 222a. In the illustrated example, the outer cover layer 222b is the outer-most cover layer, but embodiments are contemplated in which an additional cover layer is disposed on the outer surface of the outer cover layer 222b. The flexible circuit 230 is disposed between the outer surface of the inner cover layer 222a and the inner surface of the of the outer cover layer 222b and extends longitudinally along the shaft 202-1. In this embodiment, the flexible circuit 230 is attached to, such as adhered to, the inner cover layer 222a prior to the outer cover layer 222b formed on the shaft 202. In still another embodiment, the cover member (not shown) includes a single layer, and the flexible circuit is disposed between an outer surface of the support member and an inner surface of the cover member.

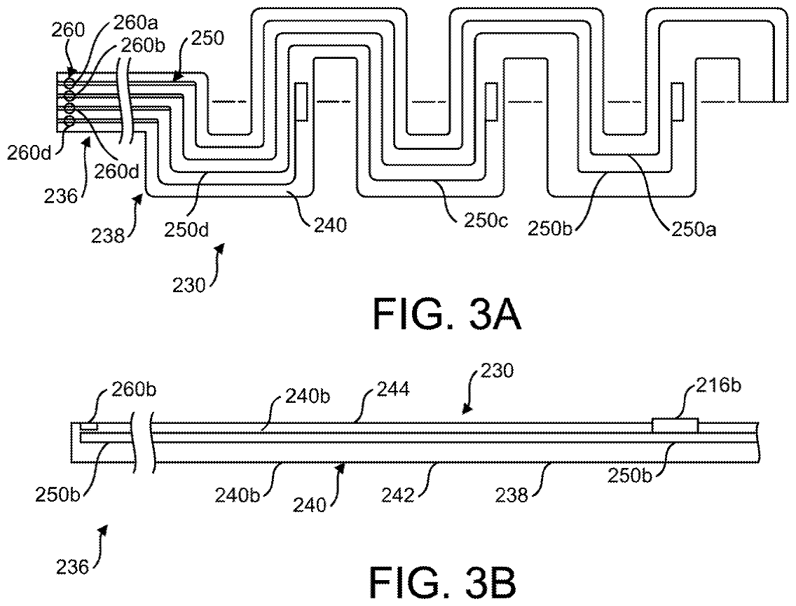

FIGS. 3A and 3B illustrate an embodiment of the flexible circuit 230. FIG. 3A illustrates a top view of the flexible circuit 230, and FIG. 3B illustrates a side view of the flexible circuit 230. The illustrated flexible circuit 230 includes a first end portion, or proximal end portion 236, configured to be at the proximal section 206 of the shaft 202 and a second end portion, or distal end portion 238, configured to be at the distal section 210 of the shaft 202. The flexible circuit 230 includes an elongated, electrically-insulative substrate 240. In some embodiments, the substrate 240 includes a plurality of electrically-insulative layers such as an underlayer 240a having a first major surface 242, configured to be a radially inward surface on the shaft 202, such as to be disposed on the outer surface of the cover member 222 in the embodiment illustrated in FIG. 2B, and an over layer 240b having opposite second major surface 244 configured to be a radially outward surface of layers 220 on the shaft 202. In one embodiment, the substrate 240 is flexible and is formed of an electrically insulative (nonconductive) material such as a thermoplastic. In another embodiment, the substrate 240 is both flexible and stretchable. Examples of a suitable material for the substrate 240 is polyimide, a Liquid Crystal Polymer (LCP), a polyether block amide (such as PEBAX), and a thermoplastic polyurethane, or TPU. In some embodiments, the substrate can include additional layers or features such as silkscreen markings. An adhesive can be applied to the first major surface 242 of the substrate 240 to attach the flexible circuit 230 to the shaft 202. In some embodiments, the adhesive layer is not present and the flexible circuit 230 is laminated to the shaft via a thermoplastic layer, such as a PEBAX Tie Layer for bonding, or via plasma treatment. In one embodiment, the thermoplastic is applied to the radially outer surface, such as the second major surface 244, to integrate the flexible circuit 230 to the shaft 202.

An electrically conductive lead trace 250 are disposed between the layers of the substrate 240, such as between layers 240a, 240b extending from the proximal, or first end portion 236 to the distal, or second end portion 238. In embodiments, the electrically conductive lead trace 250 includes a plurality of lead traces 250a . . . 250n. The plurality of lead traces 250a . . . 250n, are formed as electrical traces configured to conduct an electrical signal along the shaft 202, such as from an electrical connection at the proximal portion 206 or the handle 214 to a corresponding electrode 216a . . . 216n or electrical element such as a sensor. The number of lead traces 250a . . . 250n corresponds with at least the number of electrodes 216a . . . 216n to be attached to the shaft 202. In one embodiment, the shaft 202 includes four longitudinally spaced-apart electrodes 216a, 216b, 216c, 216d and four associated lead traces 250a, 250b, 250c, 250d. In another embodiment, the number of lead traces 250a . . . 250n corresponds with the number of electrodes 216a . . . 216n plus the number of other components electrically on the distal section 210 coupled to the proximal section 206 or handle 214. In the illustrated embodiment, the lead trace 250 terminates distally at the corresponding electrode 216. In some embodiments, components can be laminated to the substrate 240 and directly coupled to the to the lead trace 250 or to electrodes 216. In some embodiments, lead traces 250 can be provided onto substrate 240 via techniques of creating electrical circuits onto thermoset polymers or elastomers including photolithography via vapor deposition, advanced micro-electromechanical systems (MEMS) fabrication technology, flexible printed circuit board (PCB) techniques, and three-dimensional (3D) printing of conductive inks. In one embodiment, the lead trace 250 is formed as a copper tracing laminated to the substrate 240 such as between layers 240a, 240b of the substrate 240. The copper can be soft annealed to improve ductility and improved strain resistance. In some embodiments, the lead trace 250 is exposed on the substrate in portions of the flexible circuit 230 or along the entire length of the flexible circuit, such as in embodiments, in which the exposed lead traces are disposed against an electrically insulative layer of the shaft 202. In the illustrated embodiment, the lead traces 250 are electrically coupled to exposed connectors such as electrically conductive interface pads 260 at the proximal end portion 238 of the flexible circuit 230. For example, each lead trace 250a . . . 250n corresponds with an electrically connected interface pad 260a . . . 260n. The interface pads 260 are configured to be electrically coupled to wiring, circuit boards or other devices to electrically couple the lead traces 250 to the cable or the electrophysiology system 50.

In the illustrated embodiment, the electrode 216 is integrated into the flexible circuit 230. For example, the lead trace 250, such as lead trace 250b, is exposed from underneath the substrate 240 in the region of the corresponding electrode 216, such as electrode 216b. In the illustrated embodiment, the substrate 240 does not cover the lead trace 250 on the flexible circuit 230 in the region of the electrode 216, such that the electrode 216 is electrically coupled to the lead trace 250. The electrode 216 is formed of a biocompatible material such as a gold or a platinum-iridium alloy and disposed directly on the lead trace 250. In some embodiments, an iridium oxide layer is deposited on an electrically conductive material to increase the effective surface area of the electrode due to fractal surface roughness of the iridium oxide coating. Often, the effective size of the electrode 216 is larger than the width of the lead trace 240, and accordingly, portions of the electrode 216 are formed second major surface 244 of the substrate 240.

As illustrated in the plan view of FIG. 3A, the longitudinally extending flexible circuit 230, including the substrate and the lead trace 250, or plurality of lead traces 250a . . . 250n, are formed to extend along a serpentine route from the proximal end portion 236 to the distal end portion 238. In the illustrated embodiment, the serpentine route is a curvilinear serpentine route. The longitudinally extending substrate 240 is configured in the curvilinear serpentine route and the lead trace 250, or plurality of lead traces 250a . . . 250n, are configured to follow the curvilinear serpentine route of the substrate from the proximal end portion to the distal end portion 238. In the illustrated example, the curvilinear serpentine route is in the configuration of a horseshoe pattern, or as an axially extending, alternating interconnected horseshoes. The serpentine route, or pattern, in embodiments includes curvilinear serpentine routes and linear serpentine routes. Embodiments of serpentine routes include sawtooth, sinewave, and square wave patterns and are contemplated. In some embodiments, the serpentine pattern includes a trace that extends laterally back and forth, such as periodically, as it traverses the longitudinal axis. The lead trace 250 configured in a curvilinear serpentine route provides flexibility when the shaft 202 is deflected during use and enables a robust electrical connection between the proximal section 206 and the corresponding electrode 216 over, for example, purely linear longitudinal traces. In one example, the curvilinear serpentine route extends along the entire longitudinal dimension of the lead trace 250. In another example, the curvilinear serpentine route extends along selected portions of the longitudinal dimension such as the portions subjected to the most deflection during use of the shaft 202. In still another example, the amount of curvilinear serpentine routing varies, such as higher pitched serpentine routing in areas of the shaft 202 subject to more deflection and lower pitched serpentine routing in areas of the shaft 202 subject to less deflection. In one example, a higher pitched serpentine routing includes narrow horseshoes, as measured along the longitudinal axis A, and a lower pitched serpentine routing includes wider horseshoes, as measured along the longitudinal axis A.

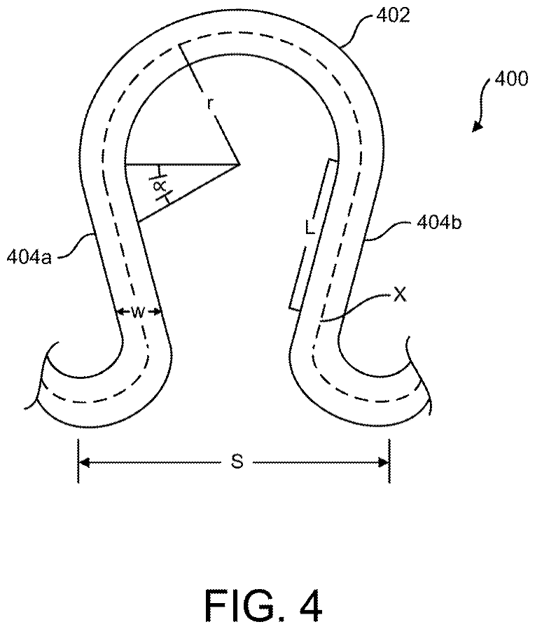

FIG. 4 illustrates a horseshoe element 400 of a lead trace 250 or a flexible circuit substrate 240 of the flexible circuit 230 configured as a horseshoe pattern along longitudinal axis A to describe geometric characteristics of the curvilinear serpentine pattern. An element 400 includes an arc 402 and a pair of arms 404a, 404b. The element is characterized by central axis x that extends through a center of the trace, a width w, such as the width of the lead trace or substrate as measured through a perpendicular of the central axis x, a radius r of the arc 402, which can be measured to the central axis x, and a length L of the arm 404a, 404b. In some embodiments, the length L of arm 404 a is equal to the length of 404b, such that the length L represents the length of either or both arms L. Further, the element includes a pitch S, which is corresponds with the number of elements per unit of length along the longitudinal axis A, and higher number of elements per unit of length along the longitudinal axis can correspond with greater strain relief properties. Additionally, a horseshoe angle α represents the angle the arc 402 extends past 180 degrees in each direction, or the angle α the end of an arc 402 extends past a line parallel with the longitudinal axis A, as illustrated. Strain experienced in the horseshoe is reduced via longer arm length L, greater horseshoe angle α, and smaller width w. For example, ratios for horseshoe pattern having effective strain relief include:

L/r≤2

w/r≤0.4

α≥0

Several examples are contemplated for flexible circuits 230 applied to shaft 202 for use in sheaths 200, such as sheaths compatible with catheter elements having an outside diameter of 8.5 F to 13 F for demonstration. A sheath having an outer diameter of around 12 F to 12.5 F are typically compatible with 8.5 F devices, and a sheath having an outer diameter of about 16 F to 17.4 F are typically compatible with 13 F devices. Greater ranges are contemplated. The width of a trace can be 15 μm or greater with a 25 μm space between traces such as traces 250 a, 250 b. In one example, the width of the trace 250 is in the range of 50 μm to 100 μm to reduce impedance or electrical resistance along the flexible circuit 230. In some embodiments, trace width along the shaft 202 is variable. For example, trace width can be increased in non-deflectable or lower strain regions of the shaft to reduce electrical resistance and narrowed in deflectable or higher strain regions. The width of the substrate 240 is reduced in size to reduce strain in the serpentine pattern. In one example, the width of the substrate can be as low small as 110 μm in circumstances with one trace 250 or two traces, one on each opposite surfaces of the substrate 240 and electrically insulated from each other. In some embodiments, traces on opposite surfaces of the substrate 240 are offset from each other, such as traces on one surface are positioned in the gaps between traces on the other surface, so as to reduce stiffness, which is increased when traces are opposite to each other on the surfaces. In examples in which the trace width is 100 μm, the width of the substrate (with four traces across the width), is in the range of 600 μm to 900 μm. Further, examples of the thickness of the substrate include a thickness of 50 μm to 80 μm or greater. Trace thickness in some embodiments is 12 μm or more. If the trace width is reduced in regions, trace thickness can be increased to compensate for changes in electrical resistance.

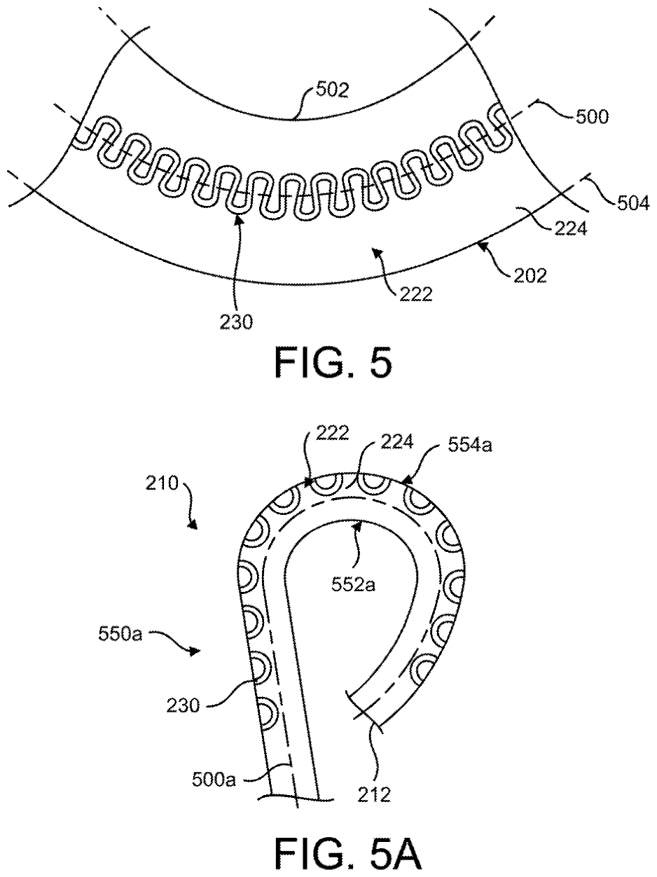

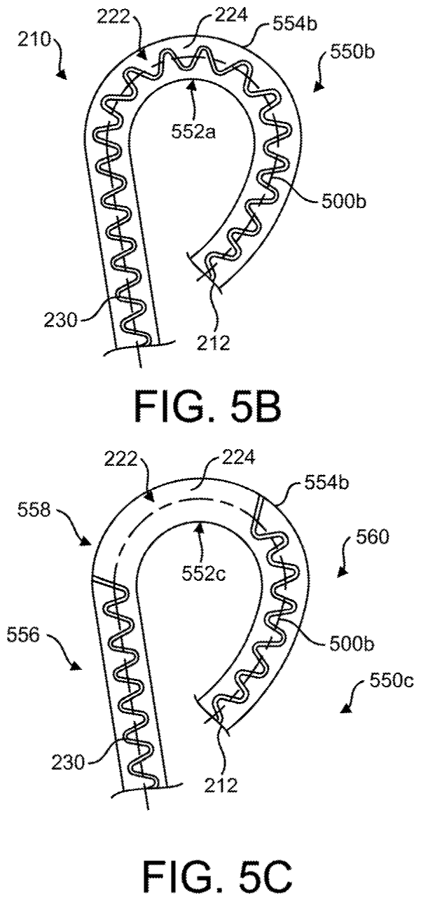

FIG. 5 illustrates an embodiment of the flexible circuit 230 positioned on the shaft 202 along a neutral axis 500 to further reduce strain in the flexible circuit 230. The neutral axis 500 is a longitudinally extending axis along the shaft 202 that is parallel with the longitudinal axis A. In the illustrated embodiment of FIG. 5, in which the flexible circuit 230 is disposed on the outer surface 224 of the cover member 222, the neutral axis 500 extends along the outer surface 224. In some embodiments, the sheath, or shaft 202, includes a deflectable region and a non-deflectable region. In a deflectable region of the sheath 200, the sheath 200 includes a compression axis 502, which extends longitudinally along the outer surface 224 of the sheath experiencing the greatest compression during a deflection, and a tension axis, which extends longitudinally along the outer surface 224 of the sheath experiencing the greatest tension and 180 degrees about the longitudinal axis A from the compression axis 502 as illustrated. In a typical sheath having a pair of opposing steering wires, the compression axis 502 can also serve as a tension axis and the tension axis 504 can serve as the compression axis depending on the direction of the deflection. Both the compression axis 502 and tension axis 552 lie in the plane of deflection. The neutral axis 500 in a sheath having a generally circular cross section is perpendicular to the plane of deflection in along a plane intersecting the longitudinal axis. Another neutral axis (not shown), or second neutral axis, is disposed along the outer surface 224 in a cross section of the shaft 202 180 degrees from the neutral axis 500, or first neutral axis, and hidden from view. The sheath at the neutral axis 500 experiences a balance of compression and tension during deflection and the neutral axis 500 is equally spaced from the compression axis 502 and the tension axis 504. In another description, the neutral axis 500 is along the outer surface 224 in a cross section of the shaft 202 90 degrees from the compression axis 502 and the tension axis 504. The balance of compression and tension during deflection is appropriate position of the flexible circuit 230 as too much compression can cause the flexible circuit 230 to delaminate or wrinkle under compression. For example, tension can lead to restriction or increase in actuation forces or tensile stresses on the flexible circuit that can cause delamination or trace cracks.

FIG. 5A illustrates an embodiment of a deflected distal section 210 of a sheath 550a, in which like parts are given like reference numbers for illustration. In the embodiment of sheath 550a, a flexible circuit 230 is disposed on the outer surface 224 of the cover member 222. The sheath 550a is illustrated as deflected in a plane of deflection and the sheath 550a includes a compression axis 552a and a tension axis 554a. The flexible circuit 230 is disposed on the outer surface 224 of the cover member 222 to extend along the tension axis 554a. In some embodiments, the distal section 210 can be articulated in a plurality of directions in the plane of deflection, and the compression axis can become the tension axis, and thus can be referred to as a compression/tension axis.

FIG. 5B illustrates an embodiment of a deflected distal section 210 of a sheath 550b, in which like parts are given like reference numbers for illustration. In the embodiment of sheath 550b, a flexible circuit 230 is disposed on the outer surface 224 of the cover member 222. The sheath 550b is illustrated as deflected in a plane of deflection and the sheath 550b includes a first compression/tension axis 552a experiencing the greatest compression in the illustrated deflection and a second, or opposite compression/tension axis 554a experiencing the greatest tension in the illustrated deflection. The flexible circuit 230 is disposed on the outer surface 224 of the cover member 222 to extend along neutral axis 500b.

FIG. 5C illustrates an embodiment of a deflected distal section 210 of a sheath 550b, in which like parts are given like reference numbers for illustration. In the embodiment of sheath 550c, a flexible circuit 230 is disposed on the outer surface 224 of the cover member 222. The sheath 550c is illustrated as deflected in a plane of deflection and the sheath 550c includes a first compression/tension axis 552c experiencing the greatest compression in the illustrated deflection and a second, or opposite compression/tension axis 554c experiencing the greatest tension in the illustrated deflection. The flexible circuit 230 illustrated as having a first portion 556 disposed on the outer surface 224 of the cover member 222 to extend along neutral axis 500b and then a second portion 558 in which the flexible circuit 230 crosses compression/tension axis 554c and extends along another neutral axis (not shown) opposite the outer surface 224 from neutral axis 500b. In some embodiments, the flexible circuit can extend in a curvilinear serpentine pattern on the another neutral axis. The flexible circuit 230 can include a third portion 560 in which the flexible circuit again crosses compression/tension axis 554c and again extends along neutral axis 500b. In some embodiments, zero or more electrodes 216 can be disposed on the first portion 556 of the flexible circuit 230, zero or more electrodes 216 can be disposed on the second portion 558 of the flexible circuit 230, and zero or more electrodes 216 can be disposed on the third portion 560 of the flexible circuit 230 in a manner such that all electrodes are disposed on the distal section 210. Patch electrodes, which do not encircle the sheath, are distinguishable from ring electrodes, which do encircle the sheath, in that patch electrodes may not be as well detected in some orientations. For example, it has been discovered that patch electrodes opposite the sheath from tracking electrical signals may not be as well detected as ring electrodes. Ring electrodes, however, do not provide information as to the orientation of the sheath or which neutral axis is toward tracking signals. To enable detection of the patch electrodes 216 in multiple orientations of the sheath 550c, one or more electrodes 216a. . . 216n are disposed on each neutral axis. Disposing electrodes 216a . . . 216n on either or both sides of the outer surface 224, such as along the neutral axes, can provide a guide as to which side of the sheath is in a visualization.

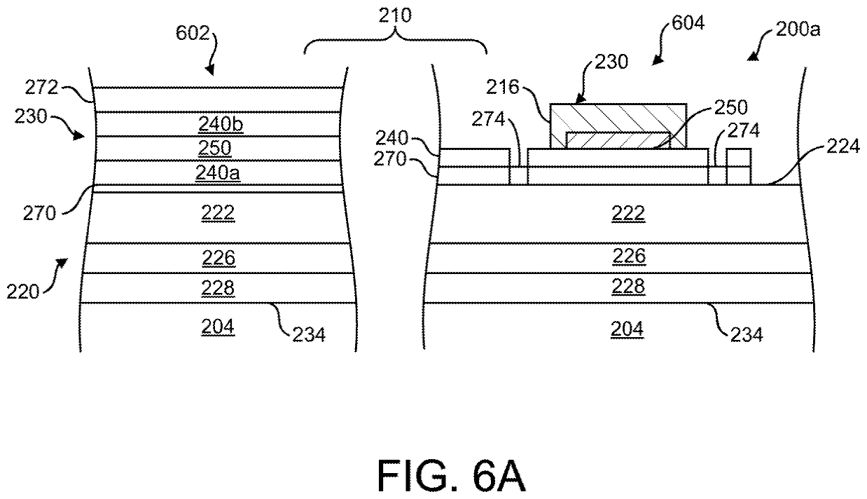

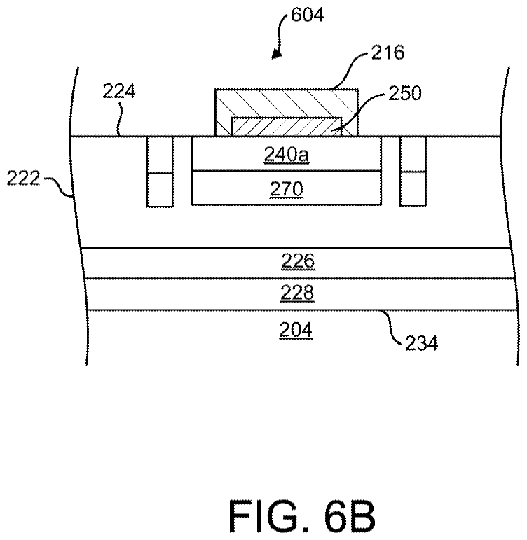

FIG. 6A illustrates a longitudinal cross section of an embodiment of a distal section 210 of a sheath 200a, in which like parts are given like reference numbers. In the illustration, the distal section 210 illustrated is a region of deflection of the sheath 200a. The illustration includes a region of the main portion 602 and a region of the electrode 604. For instance, the region of the electrode 604 is distal to the region of the main portion 602. In both the region of the main portion 602 and the region of the electrode 604, the sheath 200a includes a plurality of concentric layers 220 such as a liner layer 228 over a major lumen 204, a support member 226 radially disposed over the liner layer 228, and a cover member 222 radially disposed over the support member 226. The flexible circuit 230 is adhered to the outer surface 224 of the cover member 222. In the region of the main portion 602, the flexible circuit 230 includes a conductive trace 250 sandwiched between substrate layers 240a, 240b and attached to the outer surface 224 of the cover layer 222 such as via an adhesive layer 270 in the illustrated embodiment. In some embodiments, the adhesive layer 270 is not present. The flexible circuit 230 also includes a silkscreen layer 272 in the illustrated embodiment. At the region of an electrode 604, the overlayer 240b of the substrate 240 and the silkscreen layer 272 are removed, and an electrode 216 formed of a biocompatible material is disposed on the flexible circuit 230 over the conductive trace 250, such as covering the conductive trace 250 from exposure. The region of the electrode 604 of the illustrated embodiment also includes a plurality of micro vias 274 drilled through the thickness of the substrate.

FIG. 6B illustrates the region of the electrode 604 after the shaft 202 of FIG. 6A with a PEBAX cover member 222 has been heat staked during manufacturing. In the example, the flexible circuit 230 has been embedded into the cover member so as to be integrated under the outer surface 224. The micro vias 274 illustrated in FIG. 6A serve to integrate or anchor the flexible circuit 230 into the material of the cover member 222. A portion of the flexible circuit 230 is disposed within the cover member 222 and the electrode 216 remains exposed.

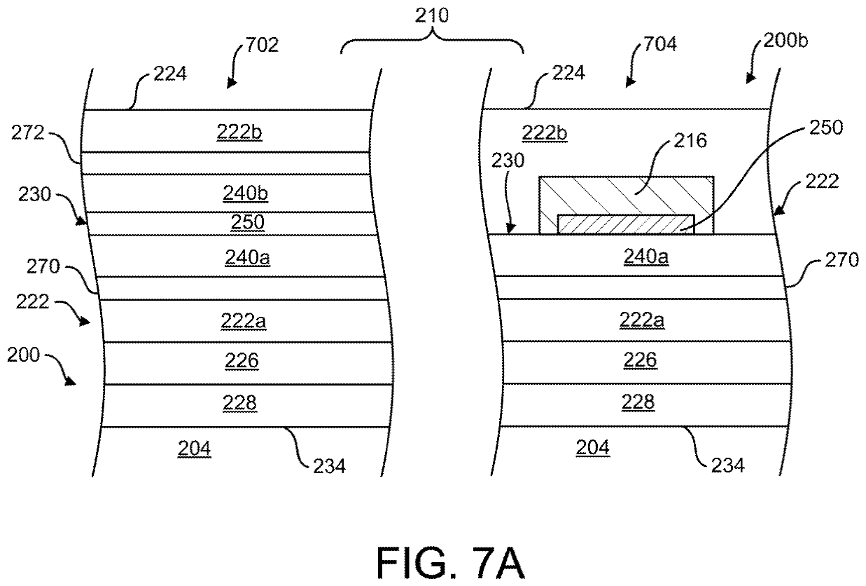

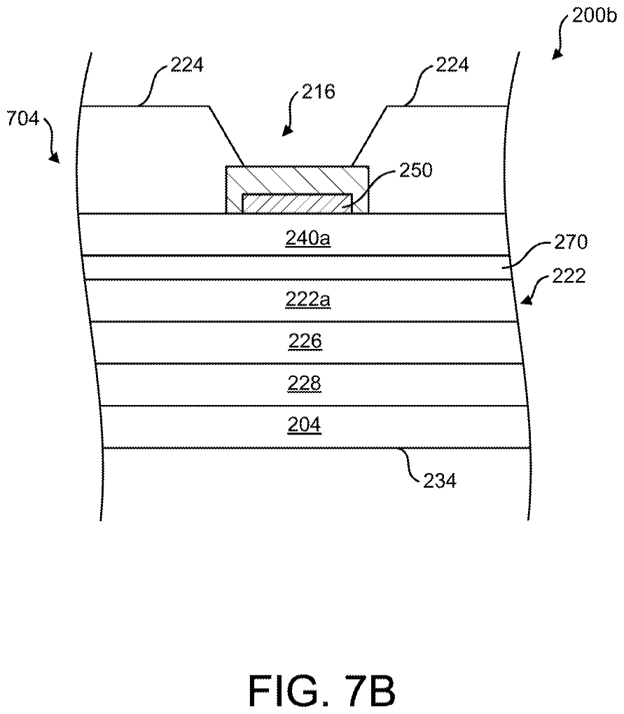

FIG. 7A illustrates a longitudinal cross section of another embodiment of a distal section 210 of a sheath 200b, in which like parts are given like reference numbers. In the illustration, the distal section 210 illustrated is a region of deflection of the sheath 200b. The illustration includes a region of the main portion 702 and a region of the electrode 704. For instance, the region of the electrode 704 is distal to the region of the main portion 702. In both the region of the main portion 702 and the region of the electrode 704, the sheath 200a includes a plurality of concentric layers 220 such as a liner layer 228 over a major lumen 204, a support member 226 radially disposed over the liner layer 228, and a cover member 222 radially disposed over the support member 226. The flexible circuit 230 is disposed underneath the outer surface 224 of the cover member 222 such as sandwiched between an inner cover layer 222a and outer cover layer 222b. In the region of the main portion 702, the flexible circuit 230 includes a conductive trace 250 sandwiched between substrate layers 240a, 240b and attached to the outer surface 224 of the cover layer 222 such as via an adhesive layer 270 in the illustrated embodiment. In some embodiments, the adhesive layer 270 is not present. The flexible circuit 230 also includes a silkscreen layer 272 in the illustrated embodiment.

FIG. 7B illustrates the region of the electrode 704 after the cover member 222 in FIG. 7A has been removed from the electrode 216 during manufacturing. At the region of an electrode 704, the overlayer 240b of the substrate 240 and the silkscreen layer 272 are not included as part of the flexible circuit 230, and an electrode 216 formed of a biocompatible material is disposed on the flexible circuit 230 over the conductive trace 250, such as covering the conductive trace 250 from exposure. The cover layer 222 radially above the electrode 216 is removed during manufacturing to expose the electrode 216 on the sheath 200b. In embodiments, the cover layer 222 is removed via mechanical or laser cutting to expose the electrode 216.

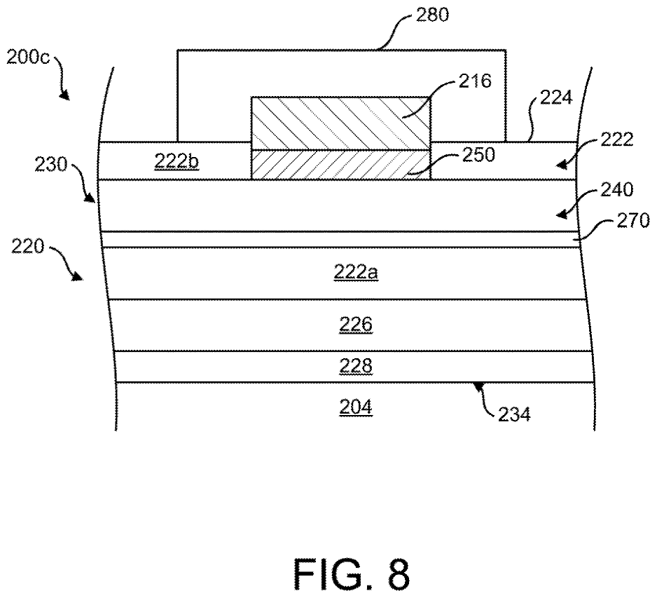

FIG. 8 illustrates a longitudinal cross section of an embodiment of a distal section 210 of a sheath 200c, in which like parts are given like reference numbers. The illustration includes a region of an electrode 804. The sheath 200c includes a plurality of concentric layers 220 such as a liner layer 228 over a major lumen 204, a support member 226 radially disposed over the liner layer 228, and a cover member 222 radially disposed over the support member 226. The flexible circuit 230 includes an electrode 216 coupled to a conductive trace 250 that is disposed on a substrate 240. The flexible circuit 230 is coupled to cover member 222 such as by an adhesive, through heat staking or other manufacturing process, and the electrode 216 is exposed from the cover member 222 if it was covered during manufacturing. The illustrated embodiment includes a ring electrode 280 electrically coupled to the electrode 216 of the flexible circuit 230. In one embodiment, the ring electrode 280 is disposed circumferentially around the shaft 202. The ring electrode 280 can be applied in circumstances in which the electrode 216 of the flexible circuit 230 is not effective enough for use with the selected impedance tracking of the EAM system 70. The ring electrode 280 is configured from a biocompatible and electrically conductive material, such as a platinum-iridium alloy, and swaged over the exposed electrode 216 of the flexible circuit 230 during manufacturing. The mechanical compression of the swaged ring electrode 280 makes the electrical connection with the electrode 216 of the flexible circuit 230 in some embodiments. In some embodiments, the ring electrode 280 and electrode 216 are welded together, such as via a laser or resistance weld.

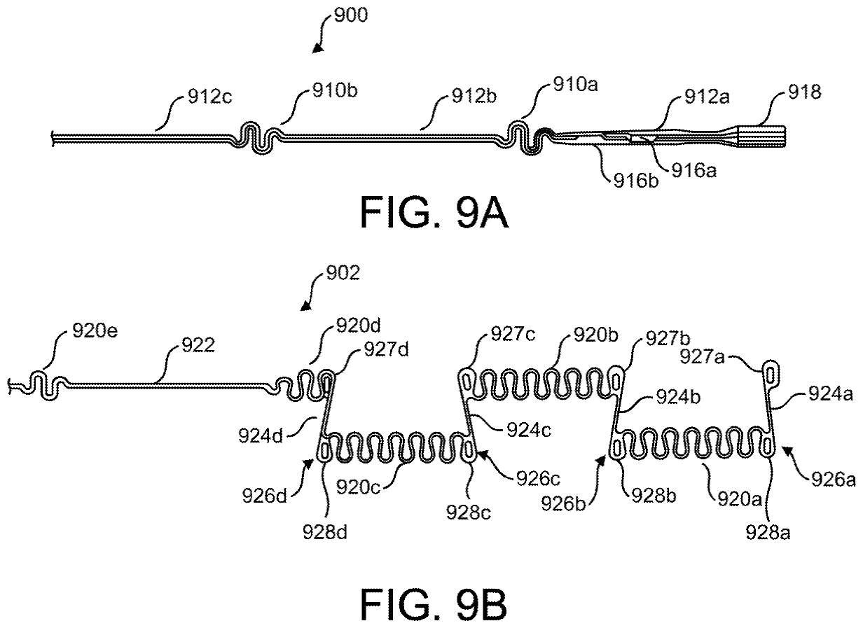

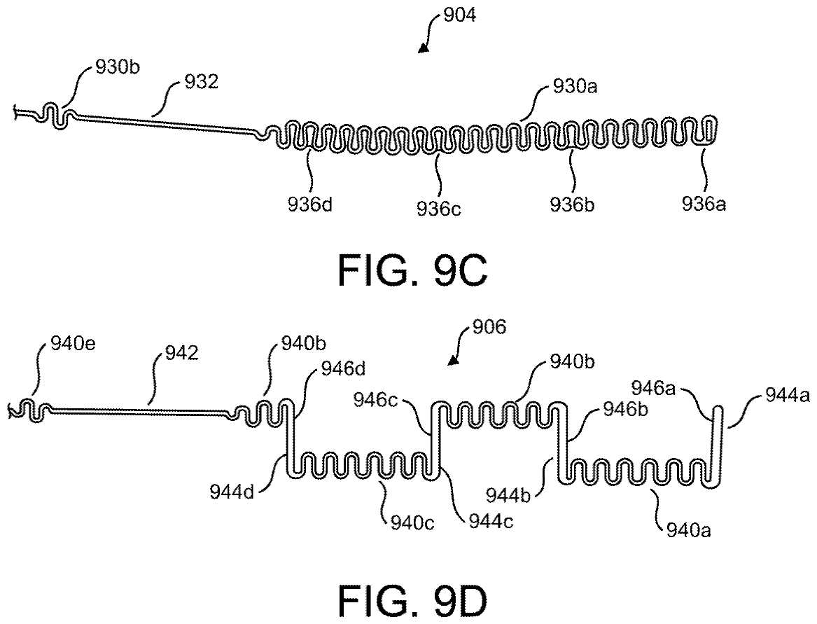

FIGS. 9A-9D illustrate distal end portions of various embodiments 900, 902, 904, 906 corresponding with the distal end portion 238 of the longitudinally extending flexible circuit 230 having the substrate 240, lead traces 250, and electrodes 216 in FIG. 2A. Other embodiments are contemplated.

FIG. 9A illustrates a distal end portion of flexible circuit embodiment 900 having a plurality of curvilinear serpentine structures 910a, 910b, in which substrate and lead traces extend laterally back and forth in a horseshoe pattern, interposed between longitudinally extending straight structures 912a, 912b, 912c, in which the substrate and traces extend longitudinally. Electrodes 916a, 916b are formed in the longitudinally straight structure 912a. The flexible circuit embodiment 900 is terminated with an electrical connection 918 configured to be connected to a sensor or other electrical component having a lead trace on the substrate. The flexible circuit 900 can be disposed along the neutral axis and between the axes of tension and compression. In one embodiment, the electrodes are disposed in the non-deflectable region. In some embodiments, the sheath, or shaft 202, for use with the flexible circuit embodiment 900 includes a deflectable region and a non-deflectable region. The straight structures, or straight routes can extend along the non-deflectable regions and the serpentine structures or serpentine routs can extend along the deflectable regions.

FIG. 9B illustrates a distal end portion of flexible circuit embodiment 902 having a plurality of curvilinear serpentine structures 920a, 920b, 920c, 920d, 920e, of varying length and number of horseshoe elements. A longitudinally extending straight structure 922 is interposed between curvilinear serpentine structures 920d and 920e. Additionally the flexible circuit embodiment 902 includes a plurality of laterally extending straight structures 924a, 924b, 924c, 924d having a length configured to cross an axis of tension/compression such that curvilinear serpentine structures 920a, 920c are wrapped around a shaft to be disposed on opposite neutral axes to curvilinear serpentine structure 920b, such as in FIG. 5C. The flexible circuit embodiment includes electrodes 926a, 926b, 926c, 926d to correspond with electrodes 216 of flexible circuit 230. In the illustrated embodiments, each electrode 926 includes a plurality of electrically conductive pads 927, 928 electrically coupled together via trace on the embodiment of the flexible circuit. For example, electrode 926a includes components 927a, 928a, electrode 926b includes components 927b, 928b, electrode 926c includes components 927c, 928c, and electrode 926d includes components 927d, 928d. The flexible circuit embodiment 902 includes an example of electrodes 926 in which the components are easily detectable with a tracking system regardless of the orientation of the sheath by disposing electrode components 927, 928 on both neutral axes. The components 927, 928 are included on portions of the flexible circuit that are on opposite neutral axes, for example, to provide for enhanced trackability via EAM system 70. In some examples, the flexible circuit embodiment 902 can provide an indication as to the orientation of the sheath and whether the sheath is twisted about the longitudinal axis based on such determinations as whether component 927a or 928a, for example, is better detected by EAM system to determine which component 927a, 928a is facing tracking signal and thus which component is opposite the sheath from the tracking signal. Electrodes can also be disposed in the non-deflectable regions of the shaft.

FIG. 9C illustrates a distal end portion of flexible circuit embodiment 904 having a plurality of curvilinear serpentine structures 930a, 930b of varying length and number of horseshoe elements. A longitudinally extending straight structure 932 is interposed between the curvilinear serpentine structures 930a, 930b. Curvilinear serpentine structure 930a is configured to extend along the deflectable portion of the sheath and includes all four electrodes 936a, 936b, 936c, 936d. The electrodes are formed as electrically conductive pads disposed within the horseshoe elements, such as between the legs.

FIG. 9D illustrates a distal end portion of flexible circuit embodiment 906 having a plurality of curvilinear serpentine structures 940a, 940b, 940c, 940d, 940e, of varying length and number of horseshoe elements. A longitudinally extending straight structure 942 is interposed between curvilinear serpentine structures 940d and 940e. Additionally the flexible circuit embodiment 906 includes a plurality of laterally extending straight structures 944a, 944b, 944c, 944d having a length configured to cross an axis of tension/compression such that curvilinear serpentine structures 940a, 940c are wrapped around a shaft to be disposed on opposite neutral axes to curvilinear serpentine structure 940b, such as in FIG. 5C. The flexible circuit embodiment includes electrodes 946a, 946b, 946c, 946d to correspond with electrodes 216 of flexible circuit 230. In the illustrated embodiments, each electrode 926 includes a single electrically conductive pad on the laterally extending straight structures 944a, 944b, 944c, 944d. The electrodes 946a, 946b, 946c, 946d are configured to partially wrap around the sheath and extend to both neutral axes, for example, to provide for enhanced trackability via EAM system 70. The flexible circuit embodiment 906 includes an example of electrodes 946 in which the components are easily detectable with a tracking system regardless of the orientation of the sheath by disposing electrode surfaces on both neutral axes. In some examples, the flexible circuit embodiment 902 can provide an indication as to the orientation of the sheath and whether the sheath is twisted about the longitudinal axis.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A sheath, comprising:
an elongated shaft defining a major lumen along a longitudinal axis and having a distal section opposite a proximal section, the shaft including a flexible circuit;
the flexible circuit having a longitudinally extending electrically insulative substrate configured in a serpentine route, an electrically conductive lead trace disposed on the electrically insulative substrate along the serpentine route, and an electrode exposed on a radially outward surface of the shaft in the distal section and electrically coupled to the lead trace, the flexible circuit extending from the proximal section to the distal section of the shaft.

2. The sheath of claim 1, wherein the lead trace is copper tracing laminated on the substrate.

3. The sheath of claim 1, wherein the substrate is a flexible and stretchable thermoplastic polyurethane.

4. The sheath of claim 1, wherein the lead trace is disposed on an inner substrate layer along a portion of the flexible circuit.

5. The sheath of claim 4, wherein the lead trace is disposed between a plurality of substrate layers including the inner substrate layer and an outer substrate layer along a portion of the flexible circuit.

6. The sheath of claim 1, wherein the flexible circuit includes an interface pad in the proximal section.

7. The sheath of claim 1, wherein the shaft includes an elongated and electrically insulative cover member having the radially outward surface.

8. The sheath of claim 7, wherein the shaft includes an elongated braided support member radially disposed underneath the cover member.

9. The sheath of claim 8, wherein the braided support member includes conductive fibers.

10. The sheath of claim 8, wherein the shaft includes an elongated and electrically insulative liner layer disposed radially underneath the braided support member, the liner layer having an inner wall defining the major lumen.

11. The sheath of claim 7, wherein the cover member includes an overlayer defining the radially outward surface and inner layer radially underneath the overlayer, the flexible circuit disposed radially between the overlayer and the underlayer.

12. The sheath of claim 11, wherein the cover member is skived to expose the electrode on the radially outward surface.

13. The sheath of claim 7, wherein the flexible circuit is adhered to the cover member.

14. The sheath of claim 1, wherein the serpentine route is a curvilinear serpentine route.

15. The sheath of claim 1, wherein the sheath includes a dilator.

16. A sheath, comprising:
an elongated shaft defining a major lumen along a longitudinal axis and having a distal section opposite a proximal section, the
shaft including a flexible circuit and having a deflectable region and a non-deflectable region;
the flexible circuit having a longitudinally extending electrically insulative substrate configured in a straight route along the non-deflectable region and a serpentine route along the deflectable region, an electrically conductive lead trace disposed on the electrically insulative substrate along the serpentine route, and an electrode exposed on a radially outward surface of the shaft in the distal section and electrically coupled to the lead trace, the flexible circuit extending from the proximal section to the distal section of the shaft.

17. The sheath of claim 16, wherein the lead trace is copper tracing laminated on the substrate, the copper tracing having a variable thickness along the deflectable and non-deflectable routes.

18. The sheath of claim 16, wherein the shaft includes an elongated braided support member radially disposed underneath the cover member, the braided support member including conductive fibers, and wherein the shaft includes an elongated and electrically insulative liner layer disposed radially underneath the braided support member, the liner layer having an inner wall defining the major lumen.

19. A method for forming a sheath, the method comprising:
forming an elongated shaft defining a major lumen along a longitudinal axis and having a distal section opposite a proximal section;
forming a flexible circuit having a longitudinally extending electrically insulative substrate configured in a serpentine route, an electrically conductive lead trace disposed on the electrically insulative substrate along the serpentine route, and an electrode exposed on a radially outward surface of the shaft in the distal section and electrically coupled to the lead trace; and
attaching the flexible circuit to the shaft, the flexible circuit extending from the proximal section to the distal section of the shaft.

20. The method of claim 19, wherein the shaft includes an elongated and electrically insulative cover member having the radially outward surface, and including skiving the cover member to expose the electrode on the radially outward surface.
